(19) United States
(12) Patent Application Publication
(10) Pub. No.: US 2023/0236208 A1
(43) Pub. Date: Jul. 27, 2023

(54) RAMAN TESTING SYSTEM AND METHODS OF DETECTING PATHOGENS

(71) Applicant: Dawatek, Inc., Oak Park, IL (US)

(72) Inventors: Isaac Wadria Chadri, Oak Park, IL (US); Davina Gali Moossazadeh, Chicago, IL (US); Leela Krishna Raavi, Chicago, IL (US); Peter Jurua Chadri, Oak Park, IL (US)

(21) Appl. No.: 18/159,782

(22) Filed: Jan. 26, 2023

Related U.S. Application Data
(60) Provisional application No. 63/303,131, filed on Jan. 26, 2022.

(51) Int. Cl. G01N 33/68; G01N 21/65; G16H 50/20

(52) U.S. Cl. CPC G01N 33/6893; G01N 21/658; G16H 50/20

(57) ABSTRACT

A Raman Testing System and Method is disclosed that uses chemical analysis to detect the chemical signature of the proteins and nucleic acids associated with pathogens coupled with machine learning to adjust to existing and potential variants of the pathogen.

### BACKGROUND

This application claims priority to U.S. provisional application Ser. No. 63/303,131, filed Jan. 26, 2022, herein incorporated by reference in its entirety.

### BACKGROUND

The invention generally relates to Raman systems, and more particularly to Raman detection of pathogens.

Raman spectroscopy is the study of small shifts in the wavelength of photons, usually generated by a laser, as the photons undergo inelastic Raman scattering with molecules in various media. Interaction with different molecules gives rise to different spectral shifts, so that analysis of a Raman spectrum can be used to determine chemical composition of a sample. The very weak nature of the scattering makes Raman spectroscopy difficult to use in many circumstances, due to the Raman signal being swamped by fluorescence and other background signals.

COVID-19 is a highly pathogenic respiratory disease, which exhibited an outbreak after its first appearance in Wuhan, China in December 2019. COVID-19 is caused by a novel coronavirus namely SARS-CoV-2, which causes respiratory illness with elevated fatality rate in patients, including patients with one or more comorbidities such as obesity, hypertension and diabetes. Cases of COVID-19 in which the patient shows no symptoms of infection appear asymptomatic but may still infect or transmit the virus to the community, state, or country.

Covid Testing is too expensive with some companies charging $45 for their test kit, and hospitals charging at least $140 per test. Covid testing is too harmful and invasive with nasal swab tests, as they are uncomfortable to patients, and the swab process puts testers in harm's way due to reactions to this invasive swab. Current Covid tests cannot scale due to limitations on PCR, expensive reagents, and taking days to receive results. Covid Testing is not reliable, where the RAPID test by Abbott has a 60-80% accuracy. The $5 antigen test that $5 has an accuracy of ˜97%, and the test cannot say definitively if there is an active infection.

The present invention attempts to solve these problems as well as others.

### SUMMARY OF THE INVENTION

Provided herein are systems, methods and compositions for a Raman Testing System and Methods of detecting Pathogens.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### BRIEF DESCRIPTION OF THE DRAWINGS

In the accompanying figures, like elements are identified by like reference numerals among the several preferred embodiments of the present invention.

FIG. 1 is a graph of saliva (newsal), saliva and kefir (newsalkefir) and kefir after the nanoparticles were subtracted, plotted against each other.

FIG. 2 is a graph of saliva (newsal), saliva and kefir (newsalkefir) and kefir without nanoparticle subtraction. The differences are noticeable in 734 cm-1 and the ratio of 2 peaks right after 1300 cm-1.

FIG. 3 is a graph of Covid-19 positive (black dots) and negative (blue line) saliva samples.

FIG. 4A is a schematic showing the use of gold nanoparticles with Surface-Enhanced Raman Spectroscopy (SERS); and FIG. 4B is a graph showing the increase of the Raman signal with gold nanoparticles in SERS.

FIGS. 5A-5B are graphs showing the use of Surface-Enhanced Raman Spectroscopy (SERS) to identify and differentiate between 2 strains of influenza virus and the negative control.

The FIGS. 6A-6B are graphs showing the use of Surface-Enhanced Raman Spectroscopy (SERS) with nanoparticles to identify and differentiate between 8 strains of rotavirus and the negative control. *

FIG. 7 is a schematic of the algorithms confusion matrix.

FIG. 8A is a photograph of Raman Testing System with the Raman spectrometer, test strip, and Raspberry Pi with LED for results.

FIG. 8B is a photograph of Raman Testing System with the handheld Raman spectrometer with collection tube and vial disposed in the Raman spectrometer.

FIG. 8C is a photograph of the Raman Testing System with the Raman spectrometer attached to Raspberry Pi with 7″ display.

FIG. 9A is a photograph of the Raman Testing System with the Raman spectrometer attached to a sample holder and right-angle laser layout.

FIG. 9B is a schematic of the sample holder and the right-angle laser layout.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein. The words proximal and distal are applied herein to denote specific ends of components of the instrument described herein. A proximal end refers to the end of an instrument nearer to an operator of the instrument when the instrument is being used. A distal end refers to the end of a component further from the operator and extending towards the surgical area of a patient and/or the implant.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The Raman Testing System and Method is used to diagnose diseases and pathogens, including, but not limited to: Covid-19, meningitis, tuberculosis, and certain cancers (including oral, lung, and breast) and other pathogens listed in Table 3. Several diseases may be detected for diagnosis, including, but not limited to: Alzheimer's, leukemia, lung cancer, oral cancer, breast cancer, pancreatic cancer, meningitis, diabetes, chronic kidney disease, urinary tract infections, diabetes. Additionally, new patterns or diseases are based on the spectral data from the Raman Testing System and Method. The Raman Testing System and Method comprises a software module that is integrated into spectroscopy hardware.

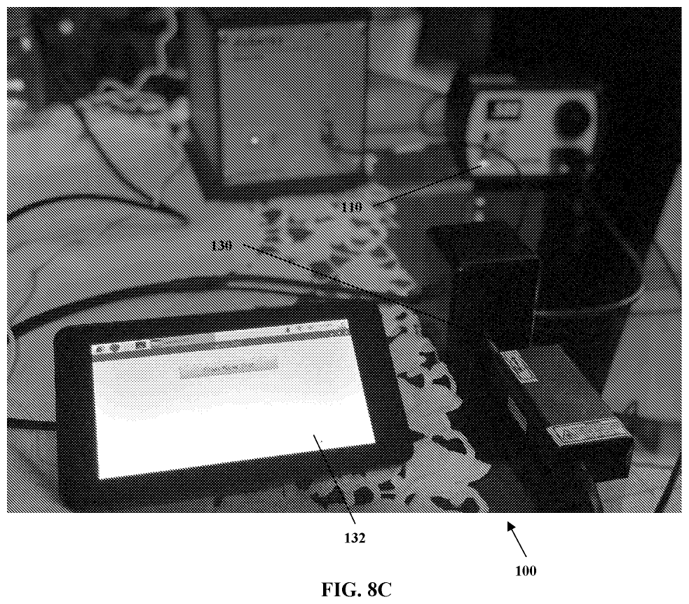

The Raman Testing System and Method samples a body fluid from a patient, where the body fluid comprises, but is not limited to: saliva, nasal mucus, blood, urine, feces, plasma, embryonic fluid, or cerebrospinal fluid. In one embodiment, the specific body fluid sampled is dependent on the disease or pathogen being diagnosed. The Raman Testing System and Method then adds to the body fluid a plurality of nanoparticles approximately the same size as the pathogen in the body fluid sample. In one embodiment, the plurality of nanoparticles are gold or silver nanoparticles. The sample comprising the body fluid and the plurality of nanoparticles is transferred to a test strip or glass vial, which is then inserted into the spectrometer for chemical analysis. FIG. 8A is a photograph of Raman Testing System 100 with a Raman spectrometer 110 operably coupled to a laser 112, a test strip 120, and a data processing system 130 with display for results.

The spectrometer produces an output of spectral data which is securely transferred to and stored into a database. The spectral data undergoes a preprocessing step by the data processing system 130 that includes a baseline correction, which comprises subtracting the nanoparticle spectra, and removing the extreme ranges of the data. Once the data is processed, the processed data is fed into a machine learning algorithm specific to the disease or pathogen being diagnosed. This Raman Testing System and Method provides a prediction of the pathogen type and load in the body fluid. The Raman Testing System and Method may also provide a prediction of the pathogen type if the machine learning algorithm differentiates between different pathogens, such as the SARS-CoV-2 and influenza viruses. The model output is stored in the database and is also used in conjunction with the spectral data, to provide a report of the pathogen load to the patient and test administrator.

Surface-Enhanced Raman Spectroscopy (SERS)

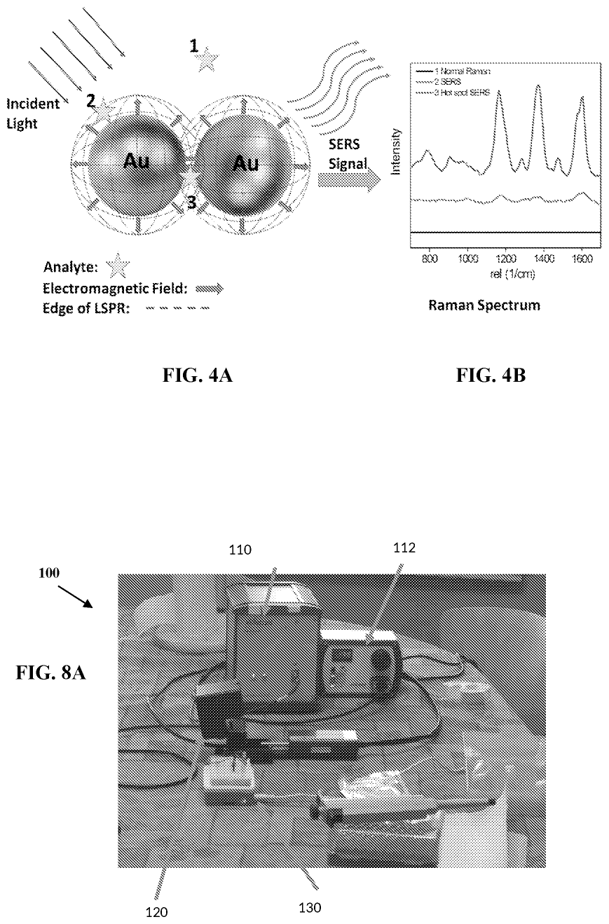

The Raman Testing System and Method comprises a collection method, a Raman spectrometer used with gold nanoparticles, and a machine learning classification model that differentiates between a sample/disease/pathogen. Raman Spectroscopy is a form of molecular analysis that is in the same wavelength range as IR, and Raman Spectroscopy works by picking up the vibrational frequencies and other low-frequency nodes in a system. The incident light excites molecular vibrations in the system leading to a shift of the scattered light which is analyzed. Thus, the Raman spectrum is fundamentally a vibrational spectrum and may be regarded as a “fingerprint” of the scattering material providing qualitative and quantitative information about the molecular composition and structure (Li-Chan, Griffith, & Chalmers, 2010; Schmidt, Scheier, & Hopkins, 2013). Raman spectroscopy is not sensitive enough to detect small biomolecules and biomarkers because the vibrational frequencies get absorbed by surrounding tissue and liquid, thus reducing the signal and making it inaccurate. Raman signals are markedly enhanced around metal nanostructures by about 106 to about 106. Furthermore, nanomaterial-based SERS can be used for molecular sensing and imaging (Lim et al. 2015). FIGS. 4A-4B show the use of gold nanoparticles with Surface-Enhanced Raman Spectroscopy (SERS) to increase the Raman signal. However, because there are only extremely small “hot-spot” in which Raman signals can be greatly enhanced, SERS has generally been used to detect molecules of less than a few nanometers in length (Lin et al., 2012, Driskell et al., 2010).

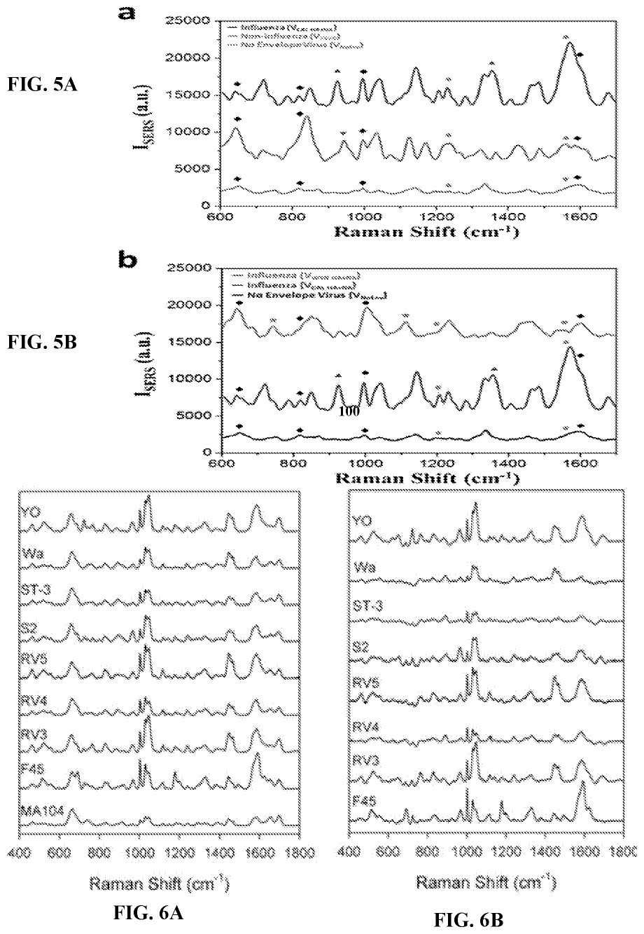

Surface-Enhanced Raman Spectroscopy (SERS) is sensitive enough to detect influenza (Van Duyne, et al. 2005). FIGS. 5A-5B shows the use of Surface-Enhanced Raman Spectroscopy (SERS) to identify and differentiate between 2 strains of influenza virus and the negative control. The influenza virus is a type of coronavirus, and is very similar in size to the novel COVID-19 virus, 80-120 nm diameter and 60-140 nm, respectively. This shows that SERS is not only able to detect COVID-19, but to also differentiate between COVID and the influenza virus.

The FIGS. 6A-6B show the use of Surface-Enhanced Raman Spectroscopy (SERS) with nanoparticles to identify and differentiate between 8 strains of rotavirus and the negative control. Table 2 is from another study looking at using SERS to differentiate between several strains of respiratory syncytial virus (RSV) with an average specificity of 95.5%.

TABLE 2
Virus strain classification based on
hierarchical cluster analysis (HCA)

| | | | Also | | |
|---|---|---|---|---|---|
| Viral | Correctly | Falsely | classified | | |
| Strain | Classified | Classified | as | Sensitivity ^{a} | Specificity ^{b} |
| RSV | 17 | 0 | — | 1.0 | 1.0 |
| A/Long | | | | | |
| RSV B1 | 17 | 0 | — | 1.0 | 0.92 |
| RSV ΔG | 15 | 2 | A2(2) | 0.88 | 0.94 |
| RSV A2 | 12 | 7 | ΔG(3), | 0.63 | 0.96 |
| | | | B1(4) | | |

^{a} Probability of correctly classifying a SERS virus spectrum as belonging to the virus strain class (i.e. a true positive)
^{b} Probability of correctly classifying a SERS virus spectrum as not belonging to the virus strain class (i.e. a negative positive)

Collection Method

For saliva collection, purification of the sample is sometimes needed in order to differentiate between samples, especially when targeting small biomarkers and proteins (Feng et al., 2015). In one embodiment, a swab saliva collection kit (Salimetrics) or a drool collection kit is used for the saliva processing, where it takes a time period between about 1 to about 2 minutes. FIG. 8B is a photograph of Raman Testing System with the handheld Raman spectrometer 102 with swab saliva collection kit 200 and vial 210 disposed of in the Raman spectrometer.

In one embodiment, the swabs serve to remove large particles of food and additional cells present in the saliva. In another embodiment, a passive drool method is used for children or infants, however, there's a chance for a sample to include unwanted components. Including the plurality of nanoparticles and a machine learning algorithm, the biomarker or pathogen will be detectable even if the saliva is not pure and includes foreign particles, food, and additional cells. For one embodiment, each subject provides about 5 ml of saliva in the morning before eating anything. In another embodiment, the subject spits into a 5 ml Eppendorf tube. For the SARS-CoV-2 method, the Salimetrics oral swabs for adults were used. About 1 ml of saliva is obtained and either about 0.5 ml or about 1 ml of saliva is used for testing. In one embodiment, the ratio of saliva to nanoparticles remains the same, so as to not alter the data. In another embodiment, an increase in volume in the saliva sample means higher intensity peaks for the results.

Raman Spectrometer

In one embodiment, the Raman Testing System and Method comprises conjugating the nanoparticles with proteins which can bind to certain receptors or markers on the target cell/molecule. However, conjugating nanoparticles with proteins to bind certain receptors or markers requires custom-made the nanoparticles and conjugation techniques, which makes it hard to replicate. In another embodiment, the Raman Testing System and Method for processes and scans samples using a Raman microscope. In another embodiment, the Raman Testing System and Method detects the diseases or pathogens in larger sizes of the body fluid sample while making it easier to replicate. In another embodiment, the Raman Testing System and Method comprises spectrometers and nanoparticles at various sizes to enhance the signal of the substance (liquid) and the target cell/molecule, then the Raman Testing System and Method uses statistical analysis paired with machine learning to identify and differentiate the samples. In one embodiment, the spectrometers are portable and/or handheld. The Raman Testing System and Method may be used as a point-of-care (POC) site with minimal technical training required and the time it takes to diagnose a disease takes minutes.

The Raman component, the Raman spectrometer, is a spectroscopic technique to determine vibrational modes of molecules, although rotational and other low-frequency modes of systems may also be observed. Raman spectroscopy relies upon inelastic scattering of photons, known as Raman scattering. A source of monochromatic light, usually from a laser in the visible, near infrared, or near ultraviolet range is used, although X-rays can also be used. The laser light interacts with molecular vibrations, phonons or other excitations in the system, resulting in the energy of the laser photons being shifted up or down. The shift in energy gives information about the vibrational modes in the system. Infrared spectroscopy typically yields similar, complementary, information. Typically, a sample is illuminated with a laser beam. Electromagnetic radiation from the illuminated spot is collected with a lens and sent through a monochromator. Elastic scattered radiation at the wavelength corresponding to the laser line (Rayleigh scattering) is filtered out by either a notch filter, edge pass filter, or a band pass filter, while the rest of the collected light is dispersed onto a detector.

In one embodiment, a standard Raman system can be used, and in other embodiments, the Raman system is a POC device handheld/portable. The Raman system is handheld and includes a vial holder that attaches to the device, as to encase the laser so no light comes out, as shown in FIG. 8B. By encasing the laser so no light escapes the Raman system, the device is safer and removes the need for safety goggles, according to one embodiment. The configuration of the device depends on the bodily fluid being examined. Saliva includes obstacles for Raman spectroscopy, so the integration time and averages have to be higher in order to get a better signal. Blood tends to absorb light in the about 700 nm wavelength which results in the laser wavelength either being lower or higher than about 700 nm to allow for signal permeation.

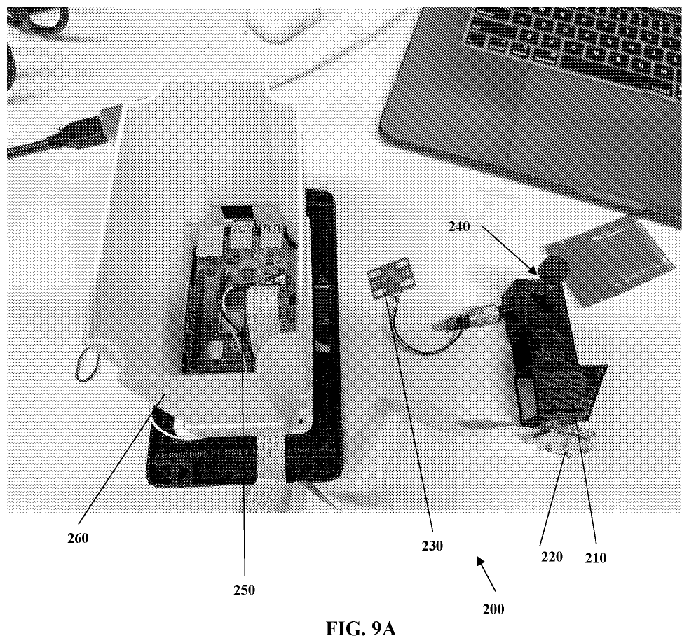

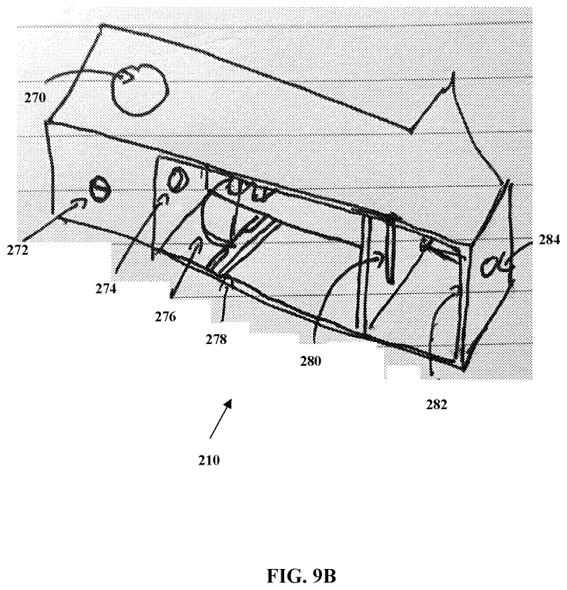

In another embodiment, the Raman system 200 includes the Raman spectrometer 210 using a NIR camera 220 and a right-angle approach for the laser 230 and a sample holder 240, the computer system 250 housed in a shell 260, as shown in FIG. 9A. As shown in FIG. 9B, first the sample goes into hole 270 which functions as the sample holder. The 532 nm laser enters through hole 272 and has a small collimating lens glued to the end of the laser. When the laser is turned on, the light will hit the sample then travel at a right angle through hole 274 and hit the large coated plano convex lens 276 (f=25.4) which focuses the light through the slit 280 where the light will spread into a spectrum. A thin diffraction grating 282 is attached to the inside wall of hole 284 and the NIR camera 220 will sit on the outer end of hole 284. The camera 220 will take a long exposure image (exposure times vary according to substance being scanned) and then is sent to the computer system 250 or the raspberry pi where its converted from an image into spectra.

In another embodiment, the spectrometer may be changed to incorporate different versions of Raman spectroscopy on top of SERS, like Spatially-Offset Raman Spectroscopy (SORS)(Matousek, 2006), Inverse Spatially-Offset Raman Spectroscopy (i SORS) (Matousek, 2007 and as disclosed in commonly assigned PCT application serial no. PCT/US2020/023991), Frequency Offset Raman Spectroscopy (FORS) (Sekar et al., 2017). Electromagnetic Field Enhancement or Chemical Enhancement may also be used for signal enhancement. These Raman techniques could be used to probe the samples at depth to get more accurate readings, especially if the samples are blood, which regular Raman is less effective at scanning.

Another embodiment can be a high throughput Raman spectrometer to scan from 2 to 100 samples at a single reading. This device may use standard Raman or transmission Raman spectroscopy to process the samples. This way all the samples can still be processed in 5 minutes.

The spectrometers may be upgraded to have computer chips capable of wireless transmission of data and Bluetooth capabilities. In one embodiment, the spectrometer and computer is one device and system.

Most spectrometers use charged coupled devices (CCD) cameras to process the light coming in. In one embodiment, each spectrometer includes Complementary Metal Oxide Semiconductor (CMOS) sensors as they are much faster at processing light through their parallel processing capabilities. (httP-s://www.teled:medalsa.com/en/learn/knowledge-center/ccd-vs-cmos/).

Data Processing

In one embodiment, the data is passed through a raspberry pi data processing system to handle the processing of the data and to display the results. FIG. 8C is a photograph of the Raman Testing System 100 with the Raman spectrometer 110 attached to Raspberry Pi data processing system 130 with 7″ display 132. In one embodiment, the data is sent to a database or server system in the cloud or internet through the Raspberry Pi instead of using the Raman spectrometer. In one embodiment, the Raspberry Pi 4 with 8 GB RAM and 7″ display screen is used to interface with both spectrometers. A more robust system may be used that's secure but functions in the same way.

Nanoparticles

As for the nanoparticle component, the type of nanoparticle being used in the Raman Testing System and Method depends on the disease or pathogen being diagnosed and what bodily fluid the pathogen is being detected in. In one embodiment, the size of the nanoparticle is about the average size of the biomarker or pathogen being detected. The type of pathogen and biomarker also affects whether gold or silver nanoparticles are used. In one embodiment, to detect presence of Lactobacillus kefir in saliva, a silver nanoparticle strips to try and detect the presence of kefir. For detecting SARS-CoV-2 in saliva, gold nanoparticles from nanocomposix that were about 80 nm in diameter and made from citrate, since the influenza virus and the Covid-19 virus are roughly the same in shape, family, and size (about 80-120 nm and about 60-140 nm in diameter, respectively). The nanoparticles are placed inside a borosilicate glass vial and when saliva samples are ready, the saliva is added to the nanoparticles, mixed for a first time period, sit still for a second time period, and then scanned. In one embodiment, the first time period is between about 2 seconds and about 30 seconds. In another embodiment, the second time period is between about 2 seconds and about 30 seconds. Borosilicate glass includes a limited small spectra that allows for detection of other types of samples without interference in detection.

In one embodiment, the nanoparticles are conjugated and used in combination or instead of standard nanoparticles for diagnosis of diseases. This embodiment may enhance the sensitivity or signal from SERS once an efficient way to manufacture the conjugated nanoparticles at scale, either in house or partnering with a manufacturer.

The mixing of conjugated and standard nanoparticles, this can be used to offer highly accurate detection along with increased overall sensitivity to notice any other patterns. A ratio of standard to conjugated nanoparticles can be 2:1, while keeping the ratio of nanoparticles to sample/liquid to 1:1, allowing for the target molecule to be detected as well as the overall spectra pattern.

In other embodiments, the type of nanoparticle can be selected from the group consisting of: gold, silver and silicone, and the subsets range from nanospheres, nanoshells (Beier et al., 2007), nanorods (Driskell et al., 2010) (https://nanocomposix.com/). Silver nanorod arrays fabricated using an oblique angle deposition (OAD) method act as extremely sensitive SERS substrates with enhancement factors of greater than 10⁸. OAD is a vapor deposition nanofabrication method that produces silver nanorods when the substrate (a smooth 500-nm silver thin film) is tilted at an 86° angle relative to the silver vapor source. The length of the nanorods increased monotonically as a function of vapor deposition time; the substrates used in the current study had an overall rod length of 868±95 nm and the diameter of the nanorods was 99±29 nm. The density of the nanorods was calculated to be 13.3±0.5 rods μm⁻² with an average tilt angle of 71±4° with respect to substrate normal. These nanorod deposition conditions were previously determined to be optimal for SERS.

Nanoshells are spherical nanoparticles with a dielectric core surrounded by a metal shell with plasmon resonances that can be adjusted by altering the ratio of the core diameter to the shell diameter. Nanoshells have been previously shown to provide significant SERS enhancements of 10⁶-10¹⁰ in the near IR by tuning the plasmon resonance to the excitation laser wavelength. Additionally, by controlling the geometry of the nanoshells, the SERS enhancement for a layer of nonresonant molecules bound to the surface of the nanoshells can be controlled with quantitative agreement between theoretical and experimental results. Solutions of nanoshells are limited by significant reabsorption of the backscattered SERS signal by other nanoshells, which limits the observed SERS enhancement. Thus, it is desired that the nanoshells be deposited onto a substrate to allow for larger SERS enhancements due to the simplified collection geometry. The intensity of the Raman signal from these nanoshell substrates has been shown to have a linear dependence on the density of nanoshells, indicating that the observed SERS signal is dependent on the resonance of single nanoshells with little contribution from aggregates.

The nanoparticles provide a nanostructured noble metal surface for Raman detection. Two dimensional silicon nanopillars decorated with silver have also been used to create SERS active substrates. The most common metals used for plasmonic surfaces are silver and gold; however, aluminium has recently been explored as an alternative plasmonic material, because its plasmon band is in the UV region, contrary to silver and gold. In the current decade, it has been recognized that the cost of SERS substrates must be reduced in order to become a commonly used analytical chemistry measurement technique. To meet this need, plasmonic paper has experienced widespread attention in the field, with highly sensitive SERS substrates being formed through approaches such as soaking, in-situ synthesis, screen printing and inkjet printing.

The shape and size of the metal nanoparticles strongly affect the strength of the enhancement because these factors influence the ratio of absorption and scattering events. There is an ideal size for these particles, and an ideal surface thickness for each experiment. Particles that are too large allow the excitation of multipoles, which are nonradiative. As only the dipole transition leads to Raman scattering, the higher-order transitions will cause a decrease in the overall efficiency of the enhancement. Particles that are too small lose their electrical conductance and cannot enhance the field. When the particle size approaches a few atoms, the definition of a plasmon does not hold, as there must be a large collection of electrons to oscillate together. An ideal SERS substrate must possess high uniformity and high field enhancement. Such substrates can be fabricated on a wafer scale and label-free superresolution microscopy has also been demonstrated using the fluctuations of surface enhanced Raman scattering signal on such highly uniform, high-performance plasmonic metasurfaces.

Chemistry Analysis

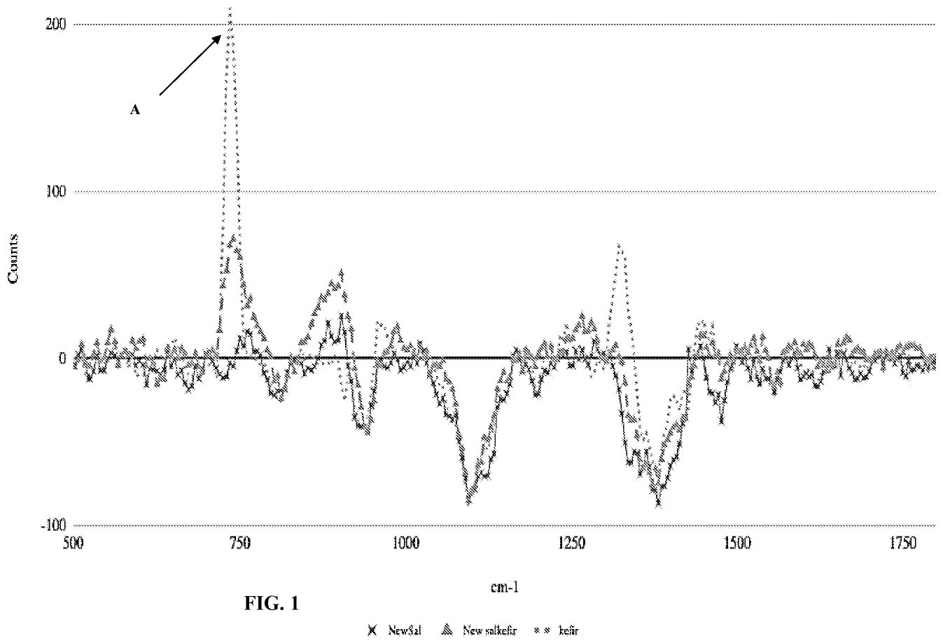

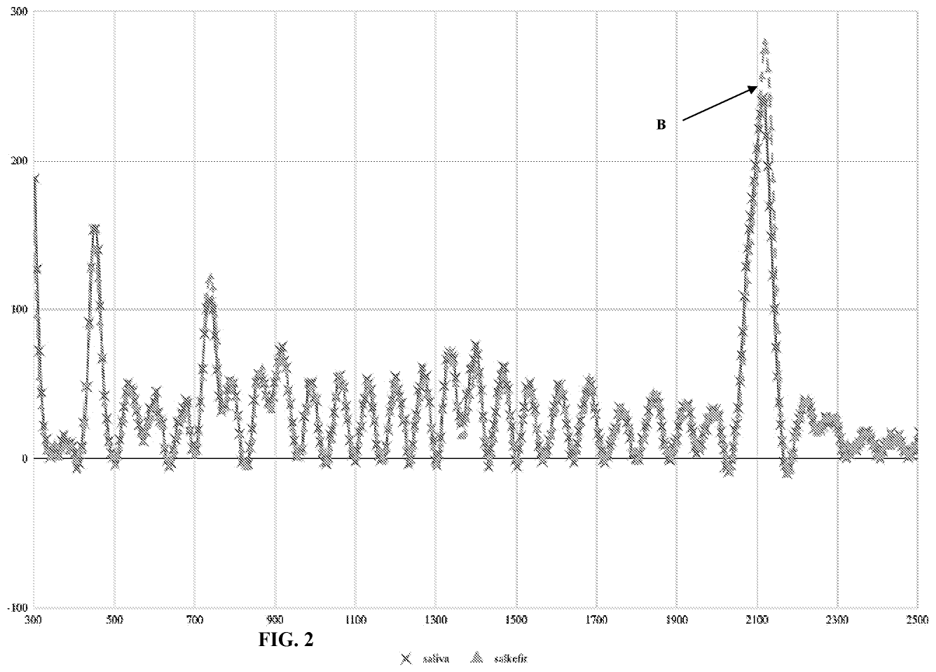

For the analysis using machine learning, the chemistry analysis comprises a spectral range to increase the accuracy. In one embodiment for the detection of bacteria within saliva, the nanoparticles are tested by themselves, the bacteria is tested alone with the nanoparticles, the saliva samples are tested by themselves, then the bacteria mixed with the different saliva samples are tested on the nanoparticle strips. Afterwards, a baseline subtraction and nanoparticle subtraction is conducted, so that the saliva (hereinafter as “sal”) and saliva+ bacteria (hereinafter as “salkefir”) looked stacked on top of each other and identify differences. FIG. 1 is a graph of subtraction of saliva and kefir, showing the peaks of kefir at 734 cm-1 and 1326 cm-1, and the salkefir samples had peaks corresponding with the kefir. The regular saliva (sal) samples did not have peaks at the designated wavelengths. Without the nanoparticle subtraction, the differences between sal and salkefir could be seen, as shown in FIG. 2, which is a graph of sal vs salkefir without subtraction, and the differences in peaks is very noticeable. The pattern is noticeable by the ratio analysis 739 cm-1 to 786 cm-1, and 1339 cm-1 to 1395 cm-1 (within −5 cm-1), which was consistent between all spectra. The next step comprises a peak assignment of relevant peaks. In one embodiment, educated guesses can be provided since there are no detailed spectras and peak analysis of 1. kefir. The known region of difference are identified, then the algorithms are reconfigured to analyze this specific section in order to get the best fit on the graph.

Kefir Bacteria in Saliva

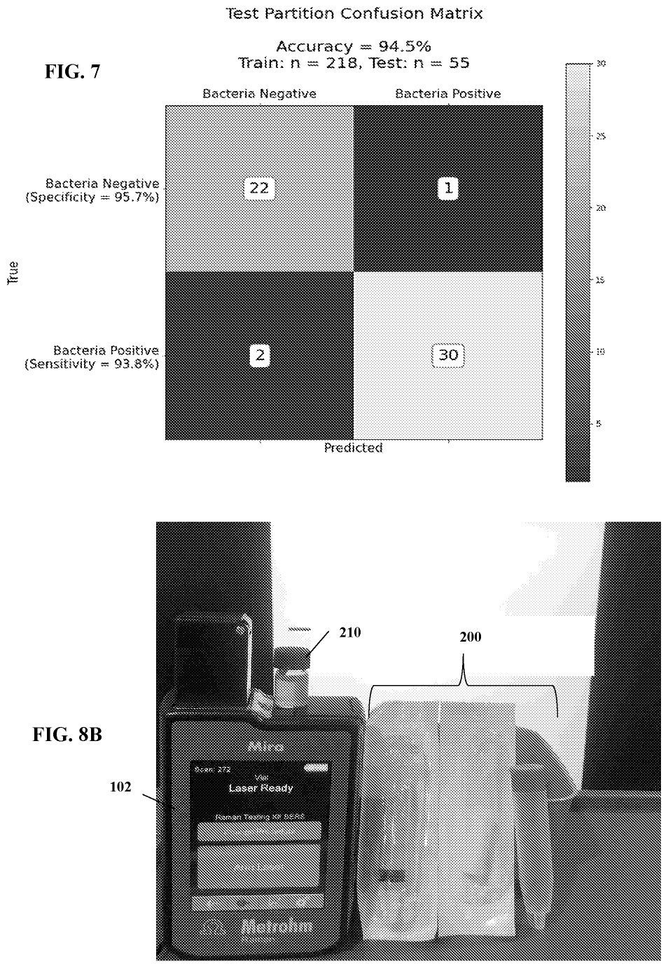

The Raman Testing System and Method analyzes saliva for presence of Lactobacillus kefir (L. kefir). The Raman Testing System and Method takes 3 scans in less than 5 minutes, 2 out of 3 (positive or negative) is the minimum requirement for diagnosis. FIG. 7 is a schematic image of the algorithms confusion matrix. Sensitivity is prioritized over specificity because it's better to have false positives rather than false negatives in disease diagnostics.

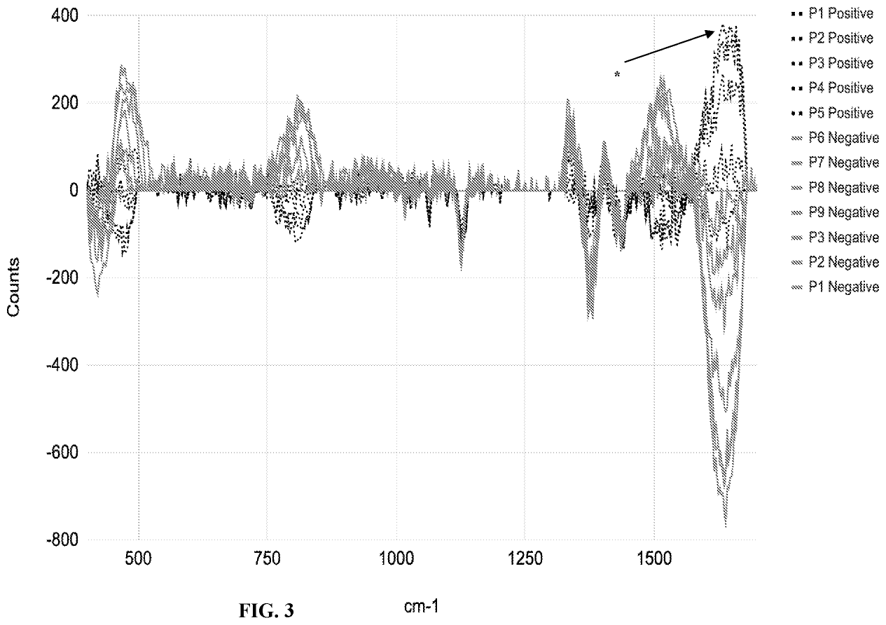

For the analysis of SARS-CoV-2, the kefir analysis and methods are replicated, according to one embodiment. In one embodiment, scans of Covid-19 negative/positive saliva samples with nanoparticles and nanoparticles with the vial only were performed. Each subject is tested the same day or a day after a PCR Covid-19 test, so as to verify whether the samples were negative or positive. After baseline correction, the vial and nanoparticles from the saliva samples are subtracted, to determine how the saliva characteristics of covid-19 positive and negative differed from each other. In one embodiment, about 7 regions were identified where negative samples differed from positive Covid-19 samples, 3 of which (marked by* in the table below) where the Covid-19 positive samples had much higher peaks than negative. This shows that these regions are where the virus is detected, and the rest correspond to biomarkers that go away while the virus is in the saliva. Peaks can be visible in FIG. 3. In another embodiment, the virus can be isolated and scanned at a microscopic level, as to ascertain what each biomarker peak corresponds to and what part of the virus is detected. Assumptions of the biomarker peaks are based on knowledge of the proteins surrounding the virus and in previous papers about saliva.

TABLE 1

| | Spectral | | Peak |
|---|---|---|---|
| Region | Range | Notes | Assignment |
| 1** | 400-450 | -All positive spectra are higher | Virus is present |
| | cm-1 | (or above) than negative ones | S-S bond |
| | | -Specific peak | |
| | | present at 421 cm-1 | |
| 2 | 450-500 | -All negative spectra are | Biomarker |
| | cm-1 | higher than positive samples | goes away |
| 3 | 750-850 | -All negative samples are | Biomarker |
| | cm-1 | higher than positive samples | goes away |
| | | -Significant peaks present | |
| | | at 772 and 800 cm-1 | |
| 4 | 1325- | -Negative samples are | Biomarker |
| | 1350 | higher than positive | goes away |
| | cm-1 | | |
| 5** | 1360- | -Positive samples are | Virus is present |
| | 1400 | higher than negative | |
| | cm-1 | -While no differentiating | |
| | | peaks present, there are | |
| | | twin peaks at 1380 and | |
| | | 1390 cm-1 that separate in | |
| | | positive samples | |
| 6 | 1460- | -Negative samples are | Biomarker |
| | 1560 | higher than positive | goes away |
| | cm-1 | -Peak shift left from | |
| | | 1545 to 1538/1540 in | |
| | | positive samples | |
| 7** | 1575- | -Positive samples are | Virus is present |
| | 1675 | higher than negative | Amide III bond |
| | cm-1 | | |

Code Description

In one embodiment, the primary coding language used in all of the software modules is python. The list of open-source python packages used in all of the modules are detailed below.

The software code can change with patient access, feedback, and control of their data in a secure way while allowing the diagnostics company to access the spectra, which can be used to improve the algorithm. The code may use Blockchain-Federated-Learning Blockchain database (Kumar et al., 2020). The data can be securely transferred from patient to hospital to the diagnosis company. The Raman Testing devices would have the capability to send the data straight to the diagnosis company.

In one embodiment, the patient stores their own medical data on the cloud server and they have the access code to it. The hospital has to request access to the data and the patient has to allow access and can remove access at any time. The only information that is sent to the diagnosis company is the patient's age, race, and gender along with the spectra. No identifying information is ever stored.

In one embodiment, the backing technology for the database may include traditional SQL, PostgreSQL, MySQL, or blockchain technology.

Spectrometer: The software to analyze the sample and produce the spectral data is provided by the partnering manufacturer. Our software provides the interface between this software and our model.

Spectral data: The data read from the spectrometer, hereinafter referred to as raw data, is output as a series of x-y coordinates, which correspond to several hundred wavenumbers of light and their respective intensities. The raw data is stored to our HIPAA-compliant database using secured transferring methods.

Data preprocessing: The raw spectral data output by the spectrometer is processed to produce the processed spectral data. This processing step involves baseline correction (subtraction of the nanoparticle spectra from the raw data) and/or limiting the spectral data range to certain wavenumbers of light.

Model Prediction: The processed spectral data is passed to the machine learning model. The specific model used for prediction depends on the disease being detected, but it can range from a simple naive Bayes' algorithm, random forest, or linear model to a deep learning or custom model. All of the models predict the load of the pathogen in question in the test sample. The output from the model is stored into the database using the secured transferring methods.

Report: The report provided to the patient and/or their provider consists of the pathogen load in the test sample. The report may also contain the raw and/or processed spectral data.

Database: The database is a HIPAA-compliant database staged on a secure server. The server may be provided by the diagnostics company or by a cloud service provider such as Amazon Web Services, Microsoft Azure, or Google Cloud Platform.

Packages

Open-source Python packages: include but are not limited to: Joblib, NumPy, Pandas, PyQt5, PyTorch, Scikit-learn, SciPy, or TensorFlow. Joblib: Used to load and save machine learning models. NumPy: Used for vectorizing, processing, and computing numerical data. Pandas: Used for handling data in tabular format. PyQt5: Used to generate graphical interface with spectrometer UI. PyTorch: Used for creating deep learning models. Scikit-learn: Used for creating classical machine learning models. SciPy: Used for scientific computing of data. TensorFlow: Used for creating deep learning models

Hardware partner package: Avaspec

Cloud service provider packages: Amazon Web Services, Microsoft Azure, or Google Cloud Platform.

Kefir and Covid-19 Testing Code

The code used for the kefir and Covid-19 tests is provided in the test.txt and functions.txt documents.

Spectrometer: Lines 39 through 148 in start_test.txt provide the interface for controlling the spectrometer, which produces the raw spectral data.

Spectral data: Lines 156, 157, and 171 in start_test.txt add the wavenumber and counts columns, and save the data.

Data preprocessing: Lines 12 through 82 in functions.txt are three methods/functions used for processing Kefir data.

The first function (baseline_correction) subtracts the nanoparticle spectra and removes the extreme ranges of the data.

The second function (find_peaks_by_width) features the spectral data by locating the highest peaks in spectral intensity and their corresponding wavenumbers.

The third function (feature creation) features the spectral data by determining ratios of specific peaks, predetermined as informative by our spectroscopic analysis.

Lines 114 and 115 in test.py filter the Kefir and Covid-19 data, respectively.

Model prediction: Lines 11 and 12 in start_test.txt load the required models into the memory.

Dimensions of the spectral intensity were reduced using Principal Component Analysis. The dimensionally-reduced data was used for modelling both kefir and Covid-19 pathogen load.

For both the kefir and Covid-19 tests, a grid search on different models and their hyperparameters was used to determine the best performing model based on the metrics of accuracy, sensitivity, and specificity. The Random Forest model was selected for kefir testing, and the Decision Tree model was selected for Covid-19 testing.

Report: File PyQt5_GUI.txt provides the interface of the software which includes starting and stopping the test, as well as displaying results. The output from model prediction is displayed by the lines 200-209 in start_test.txt.

Software Module

The purpose of the software module is to convert excel files into 2D and 3D images of grayscale. The setup generally includes a library with instructions to take a 1^{st} derivative from each spectra, storing each spectra data as a list with name of tissue, and creating a library with ‘list’ of tissues and each ‘list’ has a ‘key’. The software module then takes files from folder one at a time and goes through each file doing the following: Math Adjustments including Normalize instructions and taking the derivative. The software module then compares new graph to library and if matches, assign letter; which assigns numeric value. For a basic diagnostic, the software module repeats for every spectra it finds in folder and arranges the ‘list’ (array) in matrix shape of choice. Then with each ‘key’ in the matrix, the software module then scales the value in a 0-1 scale which is used for black and white images. For cancer detection, the software module looks at the designated cell value in all the excel file, then the value is switched to a 0-1 scale. Every new value is now put in a list then resized to fit the matrix (dimensions of scanned area). The software module then converts the matrix to an image using an RGB multiplication tool.

Software Description

As used in this application, the terms “component” and “system” are intended to refer to a computer-related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

Generally, program modules include routines, programs, components, data structures, etc., that perform particular tasks or implement particular abstract data types. Moreover, those skilled in the art will appreciate that the inventive methods can be practiced with other computer system configurations, including single-processor or multiprocessor computer systems, minicomputers, mainframe computers, as well as personal computers, hand-held computing devices, microprocessor-based or programmable consumer electronics, and the like, each of which can be operatively coupled to one or more associated devices.

The illustrated aspects of the innovation may also be practiced in distributed computing environments where certain tasks are performed by remote processing devices that are linked through a communications network. In a distributed computing environment, program modules can be located in both local and remote memory storage devices.

A computer typically includes a variety of computer-readable media. Computer-readable media can be any available media that can be accessed by the computer and includes both volatile and nonvolatile media, removable and non-removable media. By way of example, and not limitation, computer-readable media can comprise computer storage media and communication media. Computer storage media includes volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, solid state drive (SSD), or any other medium which can be used to store the desired information and which can be accessed by the computer.

Communication media typically embodies computer-readable instructions, data structures, program modules or other data in a modulated data signal such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer-readable media.

Software includes applications and algorithms. Software may be implemented in a smart phone, tablet, or personal computer, in the cloud, on a wearable device, or other computing or processing device. Software may include logs, journals, tables, games, recordings, communications, SMS messages, Web sites, charts, interactive tools, social networks, VOIP (Voice Over Internet Protocol), e-mails, and videos.

In some embodiments, some or all of the functions or process(es) described herein and performed by a computer program that is formed from computer readable program code and that is embodied in a computer readable medium. The phrase “computer readable program code” includes any type of computer code, including source code, object code, executable code, firmware, software, etc. The phrase “computer readable medium” includes any type of medium capable of being accessed by a computer, such as read only memory (ROM), random access memory (RAM), a hard disk drive, a compact disc (CD), a digital video disc (DVD), or any other type of memory.

Classification of Pathogens and Microorganism

A wide variety of pathogenic organisms may be detected by the Raman Testing System and Methods. Any type of infection within a community is likely to lead to pathogen excretion in bodily fluids/substances. Infections may be classified into symptomatic infections, and asymptomatic infections. Symptomatic infections may result in death, severe illness, moderate severity, and mild illness-all of which are clinical diseases. Asymptomatic infections may be infection without clinical illness and exposure including colonization.

Table 3 classifies pathogen and classification in categories Category A pathogens require the most intensive public preparedness efforts due to the potential for mass causalities, public fear, and civil disruption. Category B pathogens are also moderately easy to spread, but have lower mortality rates. Category C pathogens do not present a high public health threat, but could emerge as future threats

TABLE 3
The center for disease control select agents

| Category A | Category B | Category C |
|---|---|---|
| Anthrax | Brucellosis | Nipah virus |
| Bacillus anthracis | Brucella abortus | Tick-borne HFV |
| Botulism | Water and | Crimean-Congo |
| Clostridium botulinum | Food-borne | HFV |
| Coronavirus | agents | Tick-borne |
| Plague | Enteroviruses | encephalitis |
| Yersinia pestis | Poliovirus and | viruses |
| Smallpox | Rotavirus | Yellow fever |
| Variola major | Salmonellosis | Multidrug |
| Tularemia | Salmonella | resistant TB |
| Francisella | Caliciviruses | Influenza |
| tularensis | Hepatitis A virus | Flu |
| Hemorrhagic fever virus | Protozoan parasites | Other Rickettsias |
| Arenaviridae | Cryptosporidium | Rabies |
| Bunyaviridae | parvum | Zika Virus |
| Filoviridae | Giardia lamblia | |
| Flaviviridae | Toxoplasma | |
| Lassa fever | Microsporidium | |
| Hantavirus | Glanders | |
| Dengue fever | Burkholderia mallei | |
| Ebola | Psittacosis | |
| Marburg | Chlamydia psittaci | |
| Severe Acute | Q fever | |
| Respiratory Syndrome | Coxiella burnetii | |
| (SARS) | Typhus fever | |
| Parvoviruses | Rickettsia | |
| John Cunningham | prowazekii | |
| virus (JC Virus) | Viral Encephalitis | |
| Picobirnaviruses | West Nile | |
| Human Immuno- | La Crosse | |
| deficiency Virus | Venezuelan equine | |
| (HIV) | encephalitis | |
| Enteroviruses types 78-100 | Japanese | |
| Hepatitis B Virus (HBV) | encephalitis | |
| Torque teno virus (TTV) | | |

Agents causing enteric and respiratory infections are released in large numbers in saliva and respiratory secretions. Many of the enteric viruses such as the enteroviruses and adenoviruses may replicate both in the intestinal and respiratory tract. The number of enteric viruses detected can approach peak concentrations of 10¹² organisms per gram of stool while protozoa can approach 10⁶-10⁷ per gram. Cultivatable enteric bacterial pathogens such as Salmonella may also occur in concentrations as large as 10″ per gram. The concentration of respiratory viruses ranges from 10⁵ to 10⁷ per ml of respiratory secretion. Blood-borne viruses such as HIV and air-borne viruses will be found in the feces of infected persons and many viruses will occur in the urine during infection of the host, although these excreted viruses may not be infectious. The total amount of virus released by a person is, of course, also related to the amount of feces, urine, respiratory secretion, and skin that is released by the person.

In yet other aspects, the disclosed system and methods are used to analyze a blood, bodily fluid, or feces, sample and any of the following organism genera may be detected: Capnocytophaga, Rickettsia, Staphylococcus, Streptococcus, Neisseria, Mycobacterium, Klebsiella, Haemophilus, Fusobacterium, Chlamydia, Enterococcus, Escherichia, Enterobacter, Proteus, Legionella, Pseudomonas, Clostridium, Listeria, Serratia, and Salmonella and/or other bacteria, viruses, fungi, and/or protozoa. A “blood sample” may comprise blood, serum, and/or plasma.

### PUBLICATIONS

Beier H, Cowan C, Chou H, et al. Application of Surface-Enhanced Raman Spectroscopy for Detection of Beta Amyloid Using Nanoshells. Plasmonics. 2007; 55(64):55-64. DOI 10.1007/s11468-007-9027-x
Qian K, Wang Y, Hua L, Chen A, Zhang Y. New method of lung cancer detection by saliva test using surface-enhanced Raman spectroscopy. Thorac Cancer. 2018; 9(11):1556-1561. doi:10.1111/1759-7714.12837. htti:2s://www.ncbi.nlm.nih.gov/i:2mc/articles/PMC6209779/
Feng S, Huang S, Lin D, et al. Surface-enhanced Raman spectroscopy of saliva proteins for the noninvasive differentiation of benign and malignant breast tumors. Int J Nanomedicine. 2015; 10:537-547. Published 2015 Jan. 12. doi:10.2147/IJN.S71811. https://www.ncbi.nlm.nih.gov/P.,mc/articles/PMC4298339/
Falamas, Alexandra & Rotaru, Horatiu. (2020). Surface-enhanced Raman spectroscopy (SERS) investigations of saliva for oral cancer diagnosis. Lasers in Medical Science. 35. 10.1007/s10103-020-02988-2. htti:2s://www.researchgate.net/Publication/339915170.
Kaminska A, Witkowska E, Kowalska A, et al. Highly efficient SERS-based detection of cerebrospinal fluid neopterin as a diagnostic marker of bacterial infection. Anal Bioanal Chem. 2016; 408(16):4319-4327. doi:10.1007/s00216-016-9535-7. https://www.ncbi.nlm.nih.gov/Rm c/articles/PMC4875960/
Daniel, A., Prakasarao, A., David, B., Joseph, L., Murali Krishna, C., D, K. and Ganesan, S. (2014), Raman mapping of oral tissues for cancer diagnosis. J. Raman Spectrosc., 45: 541-549. https://doi.org/10.1002/jrs.4493
Falama A, Rotaru H, Hedesiu M. Surface-enhanced Raman spectroscopy (SERS) investigations of saliva for oral cancer diagnosis. Lasers Med Sci. 2020 August; 35(6):1393-1401. doi: 10.1007/s10103-020-02988-2. Epub 2020 Mar. 13. PMID: 32170505.
Lin Y Y, Liao J D, Yang M L, Wu C L. Target-size embracing dimension for sensitive detection of viruses with various sizes and influenza virus strains. Biosens Bioelectron. 2012 May 15; 35(1):447-451. doi: 10.1016/j.bios.2012.02.041. Epub 2012 Mar. 3. PMID: 22425238.
J. D. Driskell, J. L. Abell, R. A. Dluhy: Y.-P. Zhao, and R. A. Tripp “SERS-based viral fingerprinting: current capabilities and challenges”, Proc. SPIE 7703, Independent Component Analyses, Wavelets, Neural Networks, Biosystems, and Nanoengineering VIII, 770303 (12 Apr. 2010); httgs://doi.org/10.1117/12.863616
Jae-young Lim, Jung-soo Nam, Se-eun Yang, Hyunku Shin, Yoon-ha Jang, Gyu-Un Bae, Taewook Kang, Kwang-ii Lim, and Yeonho Choi. Identification of Newly Emerging Influenza Viruses by Surface-Enhanced Raman Spectroscopy. Analytical Chemistry 2015 87 (23), 11652-11659. DOI: 10.1021/acs.analchem.5b02661
Christy L. Haynes, Adam D. McFarland, and Richard P. Van Duyne. Surface-Enhanced Raman Spectroscopy. Analytical Chemistry 2005 77 (17), 338 A-346 A. DOI: 10.1021/ac053456d
Driskell J D, Zhu Y, Kirkwood C D, Zhao Y, Dluhy RA, et al. (2010) Rapid and Sensitive Detection of Rotavirus Molecular Signatures Using Surface Enhanced Raman Spectroscopy. PLOS ONE 5(4): e10222. httgs://doi.org/10.1371/journal.Rone.0010222.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A Raman Testing Method for pathogens, comprising:
Sampling a body fluid from a patient;
Adding to the body fluid a plurality of nanoparticles approximately the same size as the pathogen to be detected; and
analyzing the body fluid and the plurality of nanoparticles by a Raman spectrometer and a data processing system for chemical analysis to identify the pathogen or diagnosis disease.

2. The method of claim 1, wherein the pathogen is selected from the group consisting of:
Covid-19, meningitis, tuberculosis, and certain cancers including oral, lung, and breast.

3. The method of claim 1, wherein the disease is selected from the group consisting of:
Alzheimer's, leukemia, lung cancer, oral cancer, breast cancer, pancreatic cancer, meningitis, diabetes, chronic kidney disease, urinary tract infections, and diabetes.

4. The method of claim 3, further comprising identifying new patterns of diseases based on the spectral data.

5. The method of claim 4, further comprising integrating a software module into data processing system.

6. The method of claim 5, wherein the body fluid is selected from the group of: saliva, nasal mucus, blood, urine, feces, plasma, embryonic fluid, and cerebrospinal fluid.

7. The method of claim 6, wherein the plurality of nanoparticles are gold or silver nanoparticles.

8. The method of claim 7, wherein the Raman spectrometer produces an output of spectral data;
conducting a preprocessing step including a baseline correction, wherein the baseline correction comprises subtracting the nanoparticle spectra, and removing the extreme ranges of the data.

9. The method of claim 8, further comprising processing the data after the preprocessing step through a machine learning algorithm specific to the disease or pathogen being diagnosed.

10. The method of claim 9, further comprising predicting of the pathogen type and load in the body fluid by differentiates between different pathogens.
